(19) United States
(12) Patent Application Publication
(10) Pub. No.: US 2025/0216374 A1
(43) Pub. Date: Jul. 3, 2025

(54) METHODS AND SYSTEMS OF USING AND OPERATING PERMANENT DEFORMATION MODEL FOR SOILS AND GRANULAR MATERIALS WITH THE SHIFT FACTOR CONCEPT

(71) Applicants: PETRÓLEO BRASILEIRO S.A. – PETROBRAS, Rio de Janeiro (BR); UNIVERSIDADE FEDERAL DO RIO DE JANEIRO – UFRJ, Rio de Janeiro (BR)

(72) Inventors: Margareth CARVALHO COUTINHO CRAVO, Rio de Janeiro (BR); Bruno CAVALCANTE MOTA, Rio de Janeiro (BR); Francisco Thiago SACRAMENTO ARAGÃO, Rio de Janeiro (BR); Luis Alberto HERRMANN DO NASCIMENTO, Rio de Janeiro (BR); Mariluce DE OLIVEIRA UBALDO, Rio de Janeiro (BR)

(21) Appl. No.: 18/986,845

(22) Filed: Dec. 19, 2024

(30) Foreign Application Priority Data
Dec. 27, 2023 (BR) 1020230275230

(51) Int. Cl. G01N 33/24; G01N 3/08

(52) U.S. Cl. CPC G01N 33/24; G01N 3/08

(57) ABSTRACT

The present disclosure describes embodiments of a permanent deformation model for soils and granular materials with the application of the Shift Factor (SF) concept. The model was structured following the guidelines provided by the standard DNIT 179, but with the modification of the number of loading cycles and the stress pairs to be applied. Instead of 150,000, 10,000 load cycles were used for each stress pair, with the exception of the most severe pair, for which 150,000 load cycles continued to be applied. From the results obtained for each stress pair, there is constructed a master curve representative of one of the test stress pairs, selected as a reference. Embodiments of the present disclosure finds its field of application in the characterization of the permanent deformation of soils and granular materials.

### FIELD OF THE DISCLOSURE

The present disclosure proposes a permanent deformation model for soils and granular materials using the Shift Factor (SF) concept. The model was structured following the guidelines provided by the standard DNIT 179, but with a change in the number of loading cycles and stress pairs to be applied. Instead of 150,000, 10,000 load cycles were used for each stress pair, with the exception of the most severe pair, for which 150,000 load cycles continued to be applied. From the results obtained for each stress pair, there is constructed a master curve representative of one of the test stress pairs, selected as a reference.

The present disclosure finds its field of application in the characterization of the permanent deformation of soils and granular materials.

Although it was developed considering Brazilian materials, its applicability has no geographical limitation.

### BACKGROUNDS OF THE DISCLOSURE

In the state of the art, the permanent deformation (PD) is evaluated using triaxial repeated load equipment and consists of applying a large number of repeated load cycles (150,000 cycles) for a state of stresses in each test specimen, noting the permanent deformations accumulated over the cycles.

The correct analysis of the permanent deformation of soils and granular materials requires observing the occurrence of shakedown, which is when there is a tendency for the permanent deformation to stabilize with the number of loading cycles, for a given level of vertical and horizontal stress. Thus, in the current standard (Standard DNIT 179/18), 12 business days are required to perform the analysis of a material.

According to the DNIT guide (2006), to evaluate the quality of a highway foundation using the empirical CBR method, it is necessary to divide the subgrade into uniform segments with a maximum length of 20 km. In addition, the spacing between the drills can vary between 100 m and 200 m. Based on these guidelines, taking into account a 1 km long section and collecting data every 200 m, 5 drill holes would be necessary to perform 5 CBR analyses in the laboratory. Following this same logic for the MeDiNa method, 5 PD tests would be necessary for every 1 km of highway. This approach would result in an analysis time of 83 days, which significantly limits the geotechnical investigation of a highway engineering project.

Among the documents of the state of the art that have already addressed to the problem, the following patent and non-patent references are mentioned, for example:

The paper titled “Ensaios de deformação permanente: efeito do número de ciclos na interpretação do comportamento de solos e britas” (“Permanent deformation tests: effect of the number of cycles on the interpretation of the behavior of soils and gravel”) (Matéria Magazine, V 26, No. 03, 2021). The aforementioned document discusses concepts that are studied by researchers who address to the permanent deformation (PD) of soils and granular materials: (i) number of cycles and (ii) stopping criteria.

However, the analysis process of this paper would occur by using techniques different from those addressed to in the present disclosure, since it addresses to a technique not used in soils and granular materials, that is, the construction of master curves, the accuracy of which can be proven based on the analysis of 24 materials with different characteristics.

Caroline Dias Amancio de Lima's thesis, titled “Avaliação da deformação permanente de materiais de pavimentação a partir de ensaios triaxiais de cargas repetidas” (“Evaluation of permanent deformation of paving materials from triaxial tests of repeated loads”) (defended at COPPE/UFRJ, Rio de Janeiro, in 2020), carried out studies on the reduction of the number of load application cycles, since many authors indicated ‘different N’ as sufficient to characterize the behavior of the material.

However, embodiments of present the disclosure are not focused solely on reducing the number of test cycles, but on an optimized test and analysis process based on the construction of master curves. These master curves for soils and granular materials allowed the identification of the quantity of 10,000 cycles as being sufficient. Furthermore, although D2 had already foreseen a possibility of reducing the number of load application cycles to 10,000 initial cycles for the experimental program of the current PD standard (DNIT 179), the techniques used in D2 would be different from those described by the present disclosure because: (i) D2 does not set the quantity of 10,000 cycles for each pair in the test; and (ii) in the disclosure, the results are not cumulative, but rather translated.

The paper titled “Consideração da deformação permanente de solos ocorridos na região nordeste na análise mecanística-empírica de pavimentos” (“Consideration of permanent deformation of soils occurring in the northeast region in the mechanistic-empirical analysis of pavements”) (published in open access in 2022) already addressed to triaxial tests of repeated loads in multiple stages (applications of 10,000 cycles per stage) presenting satisfactory behavior in obtaining the PD values and using the model to predict the total PD (δp) of each material investigated in the research, by means of the correlation between the variables confining stress (σ3), shift stress (σd) and traffic (N).

However, the technique used in this document differs from embodiments of the present disclosure, because a single test specimen would be manufactured for the analysis of permanent deformation, where the deformations accumulate therein, which could impact the analysis of the permanent deformation. Differently, in the present disclosure, six test specimens are tested, one for each stress stage. Thus, the analyses and results are different.

Furthermore, the aforementioned document also does not allow analyzing the PD for a material, in a given pair of stresses, for a very high N, since the method thereof is limited to 10,000 cycles per stage. In the present disclosure, the 10,000 cycles of each stage are used in the other stages to produce master curves for analyzing the PD with N, which can reach 107, depending on the pair of stresses analyzed, increasing the observation spectrum with test data.

Larissa Montagner de Barros' thesis, titled “Implementação do ensaio Stress Sweep Rutting e do Shift Model para a previsão da deformação permanente de misturas asfálticas brasileiras” (“Implementation of the Stress Sweep Rutting test and the Shift Model to predict the permanent deformation of Brazilian asphalt mixtures”) (defended at COPPE/UFRJ, Rio de Janeiro, 2022) reports on an analysis of resistance to the accumulation of permanent deformation of asphalt mixtures using a triaxial test (Stress Sweep Rutting—SSR), the Shift Model and structural simulations using FlexPAVE™. Such a document also adopts concepts of master curve and reduced number of cycles.

However, it differs from the proposal of this patent, as the materials are completely different: asphalt mixtures are used in this one and soils and granular materials are used in the present disclosure.

In addition, the construction of the master curves of embodiments of the present disclosure was based on the principle of superposition of the load cycle-relation between stresses for the characterization of the PD of soils and granular materials, while the construction of the master curves of the aforementioned thesis was based on the principle of superposition of time for frequency) and temperature. The temperature does not influence the permanent deformation of soils and granular materials; accordingly, the concept, formulation and analysis of the PD proposed in the patent differ from that reported in the aforementioned thesis, including in relation to the applied loads, number of cycles and test specimens.

CN113326555, filed by UNIV and CHANGSHA published on Aug. 31, 2021. This document describes a rapid estimation method comprising the following steps: performing a compaction test to determine the dry density and water content of the roadbed soil; preparing a roadbed soil sample and performing a dynamic triaxial test to obtain permanent deformation values of the sample under different working conditions; establishing a pre-estimation model of the permanent deformation of the roadbed soil based on the loading state variable and the physical state variable; adjusting according to the permanent deformation value of the sample to obtain a pre-estimation model parameter, and obtaining the permanent deformation value of the roadbed soil, based on the pre-estimation model parameter and the pre-estimation model of the permanent deformation of the roadbed soil. The influence of the loading state variable and the physical state variable of the roadbed soil on the permanent deformation of the roadbed soil is comprehensively considered, the permanent deformation prediction model of the roadbed soil with wide applicability is established, and the problem that the loading state variable and the physical state variable of the roadbed soil cannot be simultaneously considered by the existing permanent deformation prediction model is solved; meanwhile, an obvious engineering convenience is provided for units without triaxial test conditions, and high market popularization value is achieved.

However, at no point does this Chinese document mention the construction of master curves or the adoption of the principle of superposition load cycle-relation between stresses to characterize the PD.

Therefore, in order to eliminate some of the difficulties present in the state of the art as mentioned above, an optimization analysis was performed for all materials that presented the overlap and the gap. Thus, it was possible to structure a test protocol, which follows the same guidelines provided by the standard DNIT 179, but with modifications to the number of loading cycles and the stress pairs to be applied.

This is because, depending on the loading frequency selected for the test, the PD test protocol—for a single material—can take twelve business days, considering the nine stress pairs specified by the standard. However, with the adoption of the PD S protocol of the master curve, proposed by the present disclosure, this time can be reduced to approximately three business days.

In the optimization analysis, it became clear that it was possible to obtain results similar to those provided by the methodology of standard DNIT 179, but with a reduction of approximately 75% in the time required to characterize the material.

Said analysis was possible due to the development of a permanent deformation model for soils and granular materials with the concept of shift factor applied to all materials that presented overlap and gap, which was structured following the guidelines provided by standard DNIT 179, but with modifications to the number of loading cycles and pairs of stresses to be applied-instead of 150,000, 10,000 load cycles were used for each stress pair. The proposed model considers the effects of confining stress and its relation with the shift stress to estimate the PD of the materials using N_{red} (number of reduced cycles) and SF (Shift factor).

### SUMMARY OF THE DISCLOSURE

The present disclosure describes a permanent deformation model for soils developed for all materials that presented overlap and gap. The proposed model considers the effects of the confining stress and its relation with the shift stress to estimate the PD of the materials using N_{red} (number of reduced cycles) and SF (Shift factor).

### BRIEF DESCRIPTION OF THE DRAWINGS

In order to make the disclosure easier to understand, there are presented by way of illustration, but without the intention of limiting the disclosure, the figures numbered 1 to 5, which accompany this specification and are an integral part of the same.

FIGS. 1a-1h represent an analysis of the PD S for the granular material M14GM, which is a modified energy gravel, where: (1a) PD of the tests carried out considering 150,000 cycles; (1b) predicted PD values for 150,000 cycles using the results of the 150,000 test cycles; (1c) predicted PD values for 150,000 cycles using 50,000 cycles extracted from the 150,000 test cycles; (1d) predicted PD values for 150,000 cycles using 25,000 cycles extracted from the 150,000 test cycles; (1e) comparison between the PD of the tests and the predictions of the PD S model for 150,000 cycles; (1f) definition of the SFs (Shift Factors) as a function of the S parameter; (1g) master curve created using 10,000 cycles of the test data in the triaxial equipment for the reference stress ratio of 40/120; and (1h) power function used in the mathematical definition of the master curve for the reference stress ratio of 40/120, according to embodiments of the present disclosure.

FIG. 2 illustrates the PD values for 150,000 cycles using the 40/120 pair for the 24 materials used as training, testing and validation of the model, according to embodiments of the present disclosure.

FIGS. 3a-3d present the results of the optimization study to evaluate redundancies and gap existing in the main PD curve, where: (3a) Initial situation (overlap and gap); (3b) Trend line for the initial situation; (3c) Optimized master curve; (3d) Trend line for the optimized master curve, according to embodiments of the present disclosure.

FIG. 4 presents an image of a dimensioned structure, where: A (black)=Coating (10 cm): RJ CAP 30/45 #12.5 mm Sepetiba; B (brown)=Base (15 cm): M16SI; and C (mustard) Subgrade: Silty soil NS′, according to embodiments of the present disclosure.

FIGS. 5a-5z present the master curves of the 24 materials tested for testing, training and validation of the use of the Shift Factor concept for predicting the permanent deformation, according to embodiments of the present disclosure.

### DETAILED DESCRIPTION OF THE DISCLOSURE

In general terms, the present disclosure describes embodiments of a permanent deformation model for soils and granular materials with the concept of Shift Factor (SF) applied to all materials that presented overlap and gap.

For information purposes, the Shift Factor (SF) is generally used in analyses or modeling to represent the impact of a change in an independent variable on a dependent variable.

The model was structured following the guidelines provided by the standard DNIT 179, but with modifications to the number of loading cycles and the pairs of stresses to be applied. Instead of 150,000, 10,000 load cycles were used for each stress pair, with the exception of the most severe pair, for which 150,000 load cycles continued to be applied. From the results obtained for each stress pair, a master curve representative of one of the test stress pairs, selected as a reference, is constructed.

The model proposed herein considered the effects of the confining stress and its relation with the drift stress to estimate the PD of the materials. The lowest confining stress and the number of load cycles were also taken into account. These parameters were also the most investigated in other models available in the literature.

It is known that, depending on the loading frequency selected for the test, the PD test protocol—for a single material—can take 12 working days, considering the nine pairs of stresses specified by the standard. With the adoption of the PD S protocol of the master curve, of the present disclosure, this time can be reduced to approximately three working days.

This reduction is very important to promote the routine characterization of these materials in field applications, which is essential for the construction of robust and long-lasting pavement structures.

Equation 1 shows the expression adopted to obtain the so-called S parameter.

$$S = {( \frac{{σ}_{3}}{0.04} )}^{{S}_{1}} \times {( \frac{{σ}_{d}}{{σ}_{3}} )}^{{S}_{2}} \qquad Equation 1$$

In Equation 1, the confining stress was divided by the lowest applied shift stress (40 kPa) and this term was raised to the regression coefficient s1. The value of 0.04 in the first term of the equation was chosen because it is the lowest applied shift stress, allowing to reduce the impacts of the inhomogeneous behavior of the granular materials.

This behavior is explained by the biophysical diversity of the microregion, that is, by the pedological, geological, vegetation, morphological and hydrological differences existing in the area where the evaluated materials are located (identified in Table 1).

Finally, the ratio between the shift and confining stresses is raised to s2 and the stress values are calculated in MPa.

With the parameter S, the PD S can then be calculated using Equation 2.

$$PD S = {a}_{1} \times {S}^{{a}_{2}} \times {N}^{{a}_{3}} \qquad Equation 2$$

The coefficients s1 and s2 of the parameter S and a1, a2 and a3 of the PD S are calculated using the Excel solver and an iterative process, to minimize the quadratic error between the deformation values obtained in the test and by the model.

The model was developed in an Excel spreadsheet, divided into five tabs: (i) PD S, (ii) Master Reference Curve, (iii) Master Curve for all pairs, (iv) Guimarães Model and (v) S.

Firstly, the data obtained in the triaxial PD test were entered. The model of the present disclosure proposed 10,000 cycles for each pair of stresses and a reduction from 9 to 6 pairs, resulting in 40/40, 40/120, 80/80, 80/240,120/240, 120/360. Only the most severe pair was subjected to 150,000 cycles to maintain the accuracy of its master curve prediction. After this step, the values of S and PD S were calculated. Next, the Excel solver was run to reduce the quadratic errors between the values obtained by PD S and the laboratory procedure.

With this procedure, the coefficients s1, s2, a1, a2 and a3 were optimized so that the PD S predictions converged with the laboratory test results. Thus, a power-type trend line could be generated, for which a descriptive equation and its coefficient of determination (R²) were identified.

Next, the optimized coefficients were transported to a second step, where the Shift Factor (SF) equal to 1 was assigned to the reference pair, and other values that shift the others to the right or left, depending on the magnitude of the pair. This process allows obtaining the master curve for a pair of reference stresses. For this, the following parameters were considered: (i) Shift Factor (SF), (ii) Reduced number of cycles (N_{red}) and (iii) S.

Unlike viscoelastic materials such as asphalt, for granular materials, the temperature does not influence the PD results. However, the magnitude of the applied confining and shift stress pairs is relevant for the characterization of the PD. Thus, analogous to the overlap principle adopted in the construction of master curves for viscoelastic properties, the superposition ‘loading cycle—magnitude of the stress pair’ is proposed for the characterization of the PD of granular materials.

In this procedure, instead of calculating reduced times or reduced frequencies, reduced cycle numbers, N_{red}, were calculated using the SFs and the real number of cycles applied in the test, N, through Equation 3:

$$N r e d = N \times SF \qquad Equation 3$$

In the procedure described herein, a pair of reference stresses was defined. For the proposal of the present model, the pair 40/120 (σc/σd, kPa/kPa) was chosen because it was applied to all the evaluated materials, being the most severe in common to all, with a ratio of ⅓.

Other stress ratios were not applied to the materials for which six stress pairs were considered, instead of the usual nine pairs, as previously mentioned.

In the shifting process, the curves of the pairs with a σc/σd ratio greater than ½ were shifted to the left, and the pairs with a ratio less than ½ were shifted to the right. This occurred because the ratios of more severe pairs (⅓) represent the permanent deformation of higher cycles for a pair with a lower stress ratio; it was found that the opposite can also occur, that is, a less severe pair with a higher stress ratio represents the permanent deformation of lower cycles.

In this process, only 10,000 cycles were needed to create the main curves, with the exception of the most severe pair, for which 150,000 load cycles continued to be applied. These curves are equivalent to those obtained by considering 150,000 cycles, presenting similar R² and power curve equation, obtained in Excel from the generation of the trend line.

This indicates that it is possible to significantly reduce the number of cycles and still obtain a good PD prediction.

In the optimization analysis, it became clear that it was possible to obtain results similar to those provided by the methodology of the standard DNIT 179, but with a reduction of approximately 75% in the time required to characterize the material, generating time and cost savings in the geotechnical prospecting step, in addition to enabling the analysis of more materials in a shorter period of time. This is because with the adoption of the PD S protocol of the master curve, this time can be reduced from 12 days to approximately three business days, without loss of accuracy.

The present disclosure can be better understood by means of the Experiments and tests that prove the results as well as the Examples described below, which are not, in any way, limiting the scope thereof, considering that there are possible additional alternatives.

### Experiments and Tests that Prove the Results

### Identification of Materials and Calculation of R²

The model coefficients were determined considering information from 24 granular materials (Table 1) that were tested to characterize their PD potential following the procedures of standard DNIT 179 of 2018.

In the compaction process, each layer is compacted with 12 strokes (Normal Energy), 26 strokes (Intermediate Energy), 39 strokes (Intermodified Energy) or 55 strokes (Modified Energy).

TABLE 1
Identification of the materials

| | Granulometric | | | |
|---|---|---|---|---|
| | composition (%) | Average | | |
| | Clay/Silt/Sand/ | MR | MCT | Compaction |
| Id. | Gravel | (MPa) | classification | energy |
| M1SN | 46/26/27/1 | 211.00 | NG′ | Normal |
| M2SN | 17/23/55/5 | 289.00 | NG′ | Normal |
| M3SI | 12/42/45/1 | 178.00 | NS′ | Intermediate |
| M4SIF | 12/42/45/1 | 241.00 | NS′ | Intermodified |
| M5SGM | soil + gravel | 313.00 | — | Modified |
| M6SN | 5/31/64/0 | 123.00 | NS′ | Normal |
| M7SN | 10/52/31/7 | 153.00 | NG′ | Normal |
| M8SN | 37/19/21/23 | 396.00 | — | Normal |
| M9SN | 17/34/44/5 | 158.05 | NS′ | Normal |
| M10SI | 3/5/35/57 | 291.50 | NA′ | Intermediate |
| M11SN | 54/7/38/1 | 201.36 | NG′ | Normal |
| M12GI | Gravel | 381.00 | — | Intermediate |
| M13GM | Gravel | 384.00 | — | Modified |
| M14GM | Gravel | 263.00 | — | Modified |
| M15GM | Gravel | 313.00 | — | Modified |
| M16SI | 56/17/27/0 | 250.00 | LG′ | Intermediate |
| M17SI | 43/23/34/0 | 302.00 | LG′ | Intermediate |
| M18SI | 1/2/13/84 | 290.00 | — | Intermediate |
| M19SI | 10/11/79/0 | 319.00 | NA′ | Intermediate |
| M20SN | 16/8/76/0 | 301.41 | NA | Normal |
| M21SN | 14/7/79/0 | 235.06 | — | Normal |
| M22SN | 23/17/59/1 | 310.00 | — | Normal |
| M23SN | 7/10/45/38 | 368.00 | — | Normal |
| M24GN | Gravel | — | — | Normal |

In order to identify the appropriate number of cycles, 150,000 load cycles were initially applied to each of the six or nine pairs of stresses, depending on the behavior of the material. These results were used to evaluate the methodology of the standard DNIT 179 and compare the same with the results of the PD S method, described herein. For this purpose, the PD of the material in 150,000 load cycles was predicted by the PD S, based on the PD obtained in laboratory tests in different numbers of cycles (25,000, 30,000, 50,000 and 150,000). Table 2 presents the R² values for when the PD prediction occurs, from the model, in relation to the triaxial test data.

TABLE 2
R² for PD S and Guimãraes Model

| | Model accuracy with test data using | | | | |
|---|---|---|---|---|---|
| | 150,000 cycles | | 50,000 | 30,000 | 25,000 |
| Material | | Guimarães | cycles | cycles | cycles |
| Id. | PD S | Model | PD S | PD S | PD S |
| M1SN | 0.8405 | 0.8381 | 0.8287 | 0.8000 | 0.7919 |
| M2SN | 0.9835 | 0.9831 | 0.9830 | 0.9807 | 0.9800 |
| M3SI | 0.5892 | 0.5913 | 0.5864 | 0.5834 | 0.5819 |
| M4SIF | 0.9714 | 0.9642 | 0.9703 | 0.9679 | 0.9669 |
| M5SGM | 0.9511 | 0.9493 | 0.9490 | 0.9444 | 0.9428 |
| M6SN | 0.9773 | 0.9768 | 0.9770 | 0.9751 | 0.9742 |
| M7SN | 0.9871 | 0.9868 | 0.9856 | 0.9819 | 0.9804 |
| M8SN | 0.9698 | 0.9690 | 0.9691 | 0.9680 | 0.9676 |
| M9SN | 0.9652 | 0.9640 | 0.9610 | 0.9500 | 0.9457 |
| M10SI | 0.9552 | 0.8611 | 0.9603 | 0.9601 | 0.9591 |
| M11SN | 0.9471 | 0.9406 | 0.9469 | 0.9431 | 0.9419 |
| M12GI | 0.6020 | 0.5989 | 0.5870 | 0.5576 | 0.5456 |
| M13GM | 0.9795 | 0.9785 | 0.9807 | 0.9803 | 0.9802 |
| M14GM | 0.9730 | 0.9724 | 0.9723 | 0.9691 | 0.9677 |
| M15GM | 0.9495 | 0.9498 | 0.9445 | 0.9386 | 0.9366 |
| M16SI | 0.9581 | 0.9572 | 0.9573 | 0.9539 | 0.9527 |
| M17SI | 0.9798 | 0.9791 | 0.9820 | 0.9819 | 0.9818 |
| M18SI | 0.9782 | 0.9780 | 0.9762 | 0.9725 | 0.9711 |
| M19SI | 0.9806 | 0.9800 | 0.9798 | 0.9776 | 0.9769 |
| M20SN | 0.9817 | 0.9816 | 0.9801 | 0.9750 | 0.9746 |
| M21SN | 0.9555 | 0.9545 | 0.9541 | 0.9496 | 0.9483 |
| M22SN | 0.9029 | 0.9013 | 0.9002 | 0.8871 | 0.8825 |
| M23SN | 0.9676 | 0.9656 | 0.9630 | 0.9554 | 0.9528 |
| M24GN | 0.9827 | 0.9825 | 0.9822 | 0.9807 | 0.9801 |

Table 2 presents the R² values obtained for different numbers of cycles adopted for the PD prediction, considering the test results.

Initially, the coefficients of the PD S model were obtained considering the PD test results for each granular material studied. The R² was calculated to quantify the accuracy of the model.

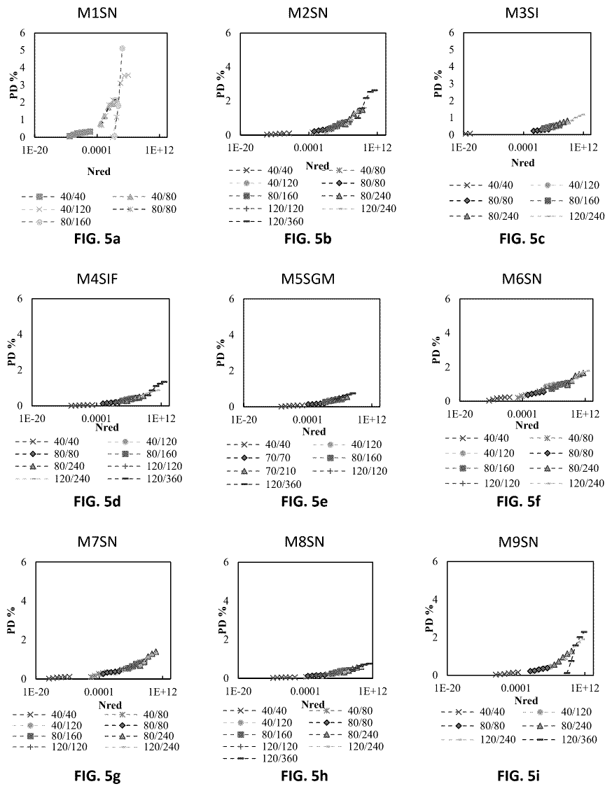

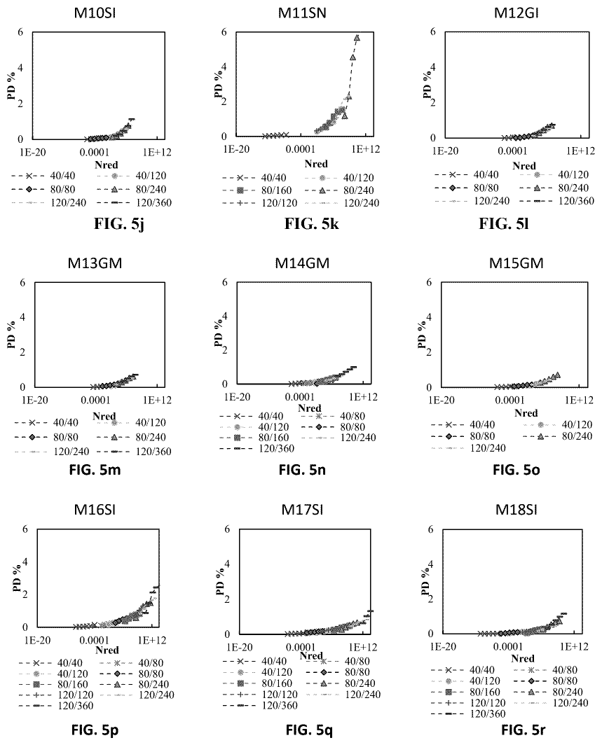

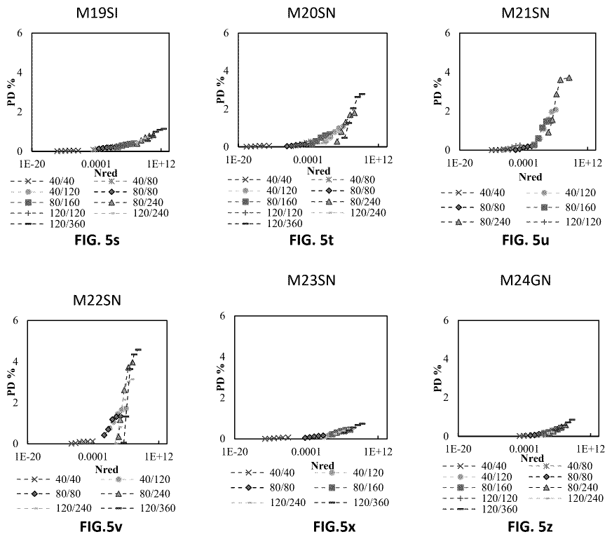

From Table 2, it is possible to verify that only the granular materials M3SI and M12GI, compacted at intermediate energy, presented low R² values. This occurred because the PD S were higher for the 40/80 stress ratio than for the 40/120 stress ratio. This can be seen in the master curves for these materials, presented in FIGS. 5a-5z.

The results in Table 2 indicated that the use of a reduced number of cycles (from 150,000 to 25,000) in the PD prediction did not affect the correlation with the results obtained in the laboratory tests. This is because the differences generally occurred in the third decimal place.

However, the adoption of 25,000 cycles would still be conflicting, as it generated overlaps between PD values for different pairs of stresses. In this sense, the number of 10,000 cycles was evaluated, identified as adequate to avoid excessive overlap of values and not compromise accuracy.

FIG. 2 presents these results, which show the PD values for three different situations: (i) triaxial test; (ii) values obtained by the Guimarães model, adopted in the standard DNIT 179 and (iii) values obtained by constructing the master curves, which are the object of this disclosure, showing that there are no disparities between the values currently adopted and those obtained by the proposal.

### Difference Between PD S Values

The test with 25,000 cycles (as described in Table 2) showed good results. However, when constructing the main curve, there were many overlapped points, including different stress pairs. Thus, after a graphical analysis, 10,000 cycles per stress pair were evaluated. The results showed that the accuracy was maintained.

Table 3 shows the percentage differences obtained by the Guimarães model and the master curve model, in relation to the test results in the triaxial equipment.

These results were obtained using only 10,000 cycles applied in the triaxial test, as proposed by the disclosure. In this sense, the master curves were constructed, which made it possible to predict the PD value for the 150,000 cycle point and, thus, compare the same with the value obtained by the current methodology of the standard DNIT 179, showing that the accuracy is maintained.

TABLE 3
Percentage difference between PD values obtained for
150,000 cycles using only 10,000 cycles (40/120)

Percentage
difference (%)

| | Test and | Test |
|---|---|---|
| Material | Guimarães | and PD |
| Id. | (2009) | S |
| M1SN | 15.26% | −16.74% |
| M2SN | −7.93% | 18.87% |
| M3SI | 27.49% | 6.44% |
| M4SIF | −43.20% | 15.12% |
| M5SGM | −12.51% | −7.12% |
| M6SN | −19.30% | −20.91% |
| M7SN | 4.68% | 13.60% |
| M8SN | 16.58% | 9.55% |
| M9SN | −11.28% | 0.29% |
| M10SI | 34.66% | 18.94% |
| M11SN | 36.78% | 19.00% |
| M12GI | −25.15% | 7.99% |
| M13GM | 5.35% | 11.45% |
| M14GM | −3.48% | −4.48% |
| M15GM | 22.19% | 17.23% |
| M16SI | −11.33% | −1.41% |
| M17SI | −26.00% | −17.69% |
| M18SI | 14.77% | 3.64% |
| M19SI | −1.22% | 2.84% |
| M20SN | −4.27% | −8.06% |
| M21SN | 21.95% | 31.04% |
| M22SN | 26.61% | 18.20% |
| M23SN | 2.03% | 1.05% |
| M24GN | 7.92% | 19.99% |

The maximum percentage difference between the PD value for 150,000 cycles, obtained by the power function equation of the master curve of the PD S model, and the value of the triaxial test, for the same number of cycles, was less than 20% for the evaluated granular materials. This value and the one next to the same were observed for only two materials.

The M21SN presented a difference above 30%. This occurred because the PD S values for the last three pairs of the curve were very close. For this material, the pair of reference stresses (40/120) presented smaller deformations than the 80/160 pair, as can be seen in FIGS. 5a-5z.

An overlap was observed in the graphs for the materials evaluated with nine stress pairs. This generated interference in the main curve. Some granular materials presented differences of less than 38. When compared with the values obtained by the Guimarães model (2009), the differences were close.

In addition, FIG. 2 presents the PD results obtained for 150,000 cycles using the Guimarães model (2009), which considers the test results for 150,000 load cycles, and using the PD S model, which requires only the results of 10,000 cycles in each pair of stresses to construct the master curve for the reference stress ratio (40/120, in this research). It is worth highlighting that some soils presented a percentage difference above 30%. However, data considered outliers were cleaned, resulting in the presented values, and only one material continued with this range.

This demonstrated the efficiency of the methodology described herein, which provides predictions similar to those obtained by the Guimarães model (2009), requiring only the characterization of the materials for 10,000 cycles at each stress rate, instead of 150,000 cycles.

### Example 1—Results for the M14GM Granular Material

### Examples

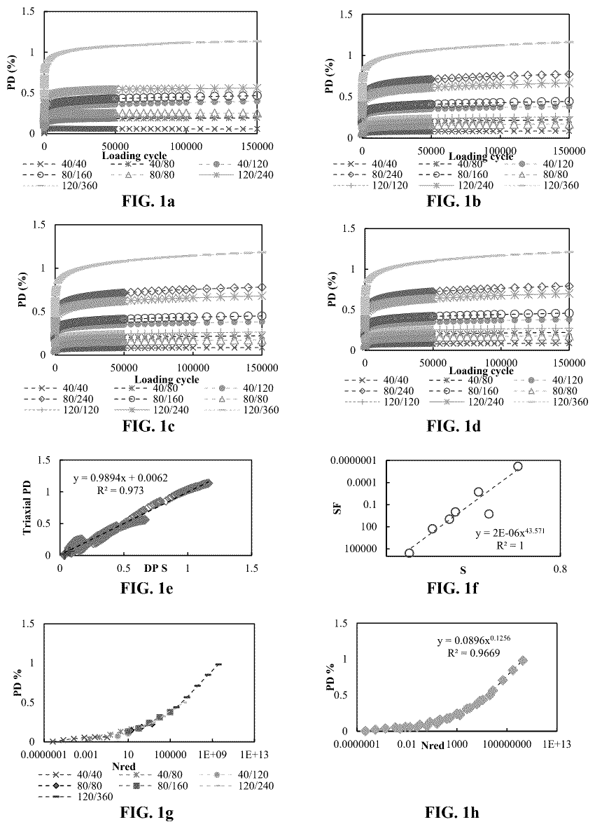

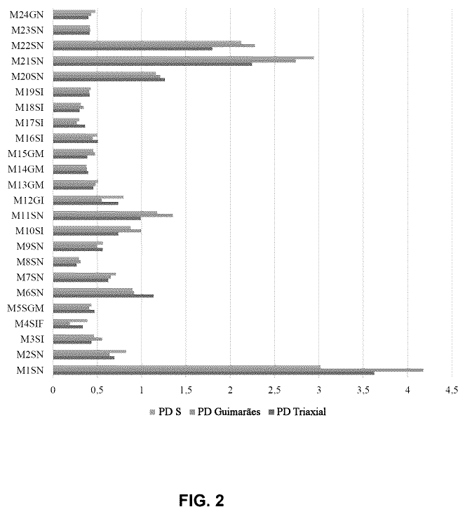

Following the described procedure, the master curve for each granular material was generated considering the results of the laboratory tests. As an illustration, FIGS. 1a-1h show the results for the M14GM granular material. In the figure, the pairs of stresses are shown as confining/shift (kPa/kPa). For example, the 120/240 case corresponds to a confining stress of 120 kPa and a shift stress of 240 kPa.

As shown in FIGS. 1a-1h and in Table 2, the R² obtained for the relation between PD S and the test results indicate that the model accurately provides PD estimates for several types of granular materials, even considering a relatively small number of load cycles.

FIGS. 1a-1d highlight the accuracy of the results shown in Table 2 for the M14GM material, as an example. FIG. 1e shows the master curve constructed for the M14GM material. FIG. 1f shows the correlation between the shift factor and the S parameter, which relates the confining and shift stresses for the M14GM material. The S parameter is used to define the shift factor for any stress rate within the evaluated range. This allows the determination of master curves for all standardized stress pairs. FIG. 1g shows the main curve for the reference stress pair, that is, 40/120 of the M14GM material, as an example. This curve can be shifted to the left or right in the reduced load cycle domain to generate master curves for other stress relations of interest. Finally, FIG. 1h presents the power function used to predict the PD for any load cycle within the N_{red} spectrum of the M14GM material, as an example.

Although the detailed curves for the other 23 materials are not presented, this example of the M14GM material was presented to provide an idea of how the process of constructing the curves and obtaining the PD values was carried out, based on the proposed disclosure. Furthermore, FIG. 5 presents the master curves for all 24 materials, following the same model as FIG. 1e.

### Example 2—Optimization for the M8SN Material

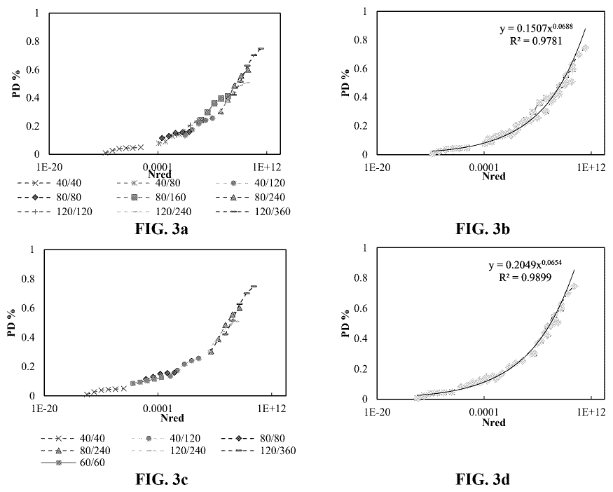

An additional evaluation was also performed regarding the overlapping between curves. In this sense, based on the observation of the graphs in FIGS. 3a-3d, the stress pairs 40/80, 80/160 and 120/120 were considered redundant, as they frequently generated significant overlaps.

The aforementioned FIGS. 3a-3d show the existence of a gap for some materials in the initial part of the master curves, after the pair of stresses 40/40. In an attempt to fill this gap, the extra pair of stresses 60/60 was evaluated, as indicated in FIG. 3c.

Although the attempt the gap was successful, the application of this extra pair of stresses can be considered unnecessary, since there was no significant change in the master curve and the incorporation of this additional pair would only increase the time required by the proposed protocol.

In this way, it can be concluded that the master curves can be constructed considering the optimization related to the removal of redundancies that generate an excessive overlap points, but without additional action to fill the gap in their initial part.

### Example 3—Sizing of the M16SI Material

Finally, sizing was performed in the MeDiNa program to observe the wheel track depression (RTA) of the materials using the Guimarães coefficients obtained with the 150,000 cycles and those obtained from the model described herein. The RTA is the consequence of permanent deformation. Permanent deformation is the ‘Residual Stress Amplitude’, which is an important parameter in fatigue studies of materials and structures, as it represents the difference between the maximum stress and the minimum stress in a repetitive load cycle.

A standard structure with coating and subgrade from the program database was evaluated. Thus, the materials used in the model studies were inserted as the base layer.

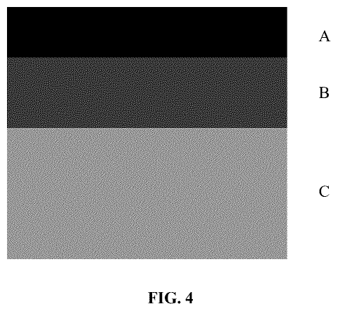

For the M16SI material, for example, three different traffic conditions were evaluated and a difference of only 5% was observed in relation to the results obtained with 150,000 cycles from those predicted by the master curves with 10,000 cycles. Table 4 presents the results for this material and FIG. 4 presents the analyzed structure.

TABLE 4
Results of the sizing of the M16SI material

| | Guimaräes Model | | | | | |
|---|---|---|---|---|---|---|
| | Total PD (mm) | | | PD Layer (mm) | | |
| | DNIT | Master | | DNIT | Master | |
| Traffic | 179 | Curve | Diff % | 179 | Curve | Diff % |
| 5.00E+05 | 1.9 | 1.9 | 0% | 0.52 | 0.54 | 4% |
| 5.00E+06 | 2.2 | 2.2 | 0% | 0.58 | 0.61 | 5% |
| 5.00E+07 | 2.5 | 2.6 | 4% | 0.65 | 0.69 | 6% |

From this analysis, it was observed that the percentage differences in the subsidence of the pavement layers considering the Guimarães model, adopted by DNIT 179, and the model considering the results obtained by the master curve, proposed by the disclosure, are very low, not causing oversizing or undersizing of the structure and demonstrating the accuracy of the proposed method.

Thus, the proposed model, although it follows the same guidelines provided by the standard DNIT 179, suggested modifications in the number of loading cycles and the pairs of stresses to be applied. The PD test protocol—for a single material-which would normally take twelve business days, considering the nine pairs of stresses specified by the standard, with the adoption of the PD S protocol of the master curve, can be reduced to approximately three business days, that is, a 75% reduction in the time required to characterize the material, generating time and cost savings in the geotechnical prospecting step, in addition to enabling the analysis of more materials in a reduced period of time.

The description made so far of the present disclosure should be considered only as a possible embodiment, and any particular features should be understood as something that was described to facilitate understanding. In this way, they cannot be considered limiting the disclosure, which is limited only to the scope of the claims that follow.

1. A method of operating a permanent deformation model to analyze one or more soils or granular materials with the shift factor concept, the method comprising:
using a triaxial test with reduced load cycles for each stress pair, from construction of a master curve for a reference stress ratio, obtained through Equations 1 and 2:
1—obtaining the parameter S
$$S = {( \frac{{σ}_{3}}{0.04} )}^{{S}_{1}} \times {( \frac{{σ}_{d}}{{σ}_{3}} )}^{{S}_{2}} \qquad Equation 1$$
2—obtaining the PD S
$$PD S = {a}_{1} \times {S}^{{a}_{2}} \times {N}^{{a}_{3}} \qquad Equation 2$$
3—obtaining the master curve for a pair of reference stresses, which considers the following parameters: (i) Shift Factor (SF), (ii) Reduced number of cycles (N_{red}) and (iii) and the real numbers of cycles applied in the test, N, through Equation 3:
$$N r e d = N \times SF ; \qquad Equation 3$$
and
characterizing permanent deformation of one or more soils or granular materials.

2. The method according to claim 1, wherein the triaxial test is used with 10,000 load cycles for each stress pair, from the master curve to the reference stress ratio, except for the most severe one that would continue up to 150,000.

3. The method according to claim 1, wherein the characterizing considers the effects of the confining stress and its relation with the shift stress to estimate the permanent deformation of the one or more soles or materials in a wide space of cycles by use of the curves of other pairs.

4. The method according to claim 1, wherein the model has reduced from 9 to 6 stress pairs, resulting in the following pairs: 40/40, 40/120, 80/80, 80/240,120/240, 120/360.

5. The method according to claim 4, wherein the stress pair 40/120 is the reference for generating the master curve.

6. The method according to claim 1, wherein the master curve for a pair of reference stresses is obtained by assigning a Shift Factor (SF) equal to 1, for the reference pair, and other values that shift the others to the right or to the left, depending on the magnitude of pair.

7. The method according to claim 1, wherein adoption of the PD S protocol of the master curve reduces the same from 12 days to approximately three working days for the characterization of a material in geotechnical prospecting.

8. The method according to claim 1, wherein the model is developed in an Excel spreadsheet, divided into five tabs: (i) PD S, (ii) Master Reference Curve, (iii) Master Curve for all pairs, (iv) Guimarães Model and (v) S.
